(19) United States
(12) Patent Application Publication
(10) Pub. No.: US 2011/0189286 A1
(43) Pub. Date: Aug. 4, 2011

(54) Pulsatile Release of Valsartan

(76) Inventors: Amol Singh Matharu, Bedminster, NJ (US); Agnes Taillardat, Seewen (CH)

(21) Appl. No.: 12/995,577

(22) PCT Filed: Jun. 1, 2009
(86) PCT No.: PCT/US09/45786

§ 371 Date: Apr. 25, 2011

Related U.S. Application Data
(60) Provisional application No. 61/058,233, filed on Jun. 3, 2008.

(51) Int. Cl. A61K 31/41; A61K 9/00; A61P 9/00; A61K 9/10

(52) U.S. Cl. 424/484; 514/381; 424/400; 427/2.19

(57) ABSTRACT

The present invention provides gastroretentive pulsatile pharmaceutical delivery systems that improve the bioavailability of Valsartan wherein the medicament has improved solubility, improved residence time in the gastrointestinal tract and a pulsatile release profile.

### BACKGROUND

Angiotensin II is a very potent end product chemical that causes the muscles surrounding the blood vessels to contract, thereby significantly narrowing those vessels. This narrowing increases the pressure within arterial vessels, causing high blood pressure (hypertension). Angiotensin receptor blockers (ARBs) are drugs that block the action of angiotensin II. As a result, arterial vessels dilate and blood pressure is reduced, thereby making it easier for the heart to pump blood. ARBs can therefore also be used to improve heart failure, as well as hypertension. In addition, they slow the progression of kidney disease due to high blood pressure or diabetes.

Valsartan is an important ARB, the synthesis and use of which are described in U.S. Pat. No. 5,399,578, which is incorporated herein by reference in its entirety. However, Valsartan has poor disintegration and solubility and consequently has low bioavailability. The low bioavailability associated with poor aqueous solubility warrants administration of larger doses of Valsartan, delivered in a controlled release manner, to maintain desired therapeutic activity.

Conventional controlled release drug delivery systems have only limited use for (1) drugs having a narrow absorption window in the gastrointestinal tract, i.e., are absorbed in the duodenum and/or jejunum; (2) local treatment of proximal parts of the gastrointestinal tract (stomach and/or duodenum); and (3) drugs that degrade in the colon.

According to the basic principle of drug absorption, only the drug in the neutral form present in solution can permeate across the lipid cell membranes. Therefore, for a better absorption, the drug substance should be lipophilic in nature and have adequate solubility in the GI milieu. Valsartan, for example, has a free carboxylic acid group, which makes it insoluble in acidic conditions and ionized (soluble form) in alkaline environments. Absorption of Valsartan in an acidic environment is therefore low due to its poor solubility. By contrast, in an alkaline environment, Valsartan is in the ionized (soluble) form and thus has low lipophilicity and consequently has poor cell membrane permeation. In other words, Valsartan has poor absorption in the gastrointestinal tract either due to a combination of poor solubility of the free acid form in acidic/weakly acidic GI milieu and poor permeability of the dissolved (ionized) form. The result of this low solubility and low permeability is low bioavailability of 10-25%.

Furthermore, the transit time through the gastrointestinal tract often limits the amount of drug available for absorption at its most efficient absorption site. As the solubility of the drug decreases, the time required for drug dissolution and absorption through the intestinal membrane becomes less adequate and, thus, the transit time becomes a significant factor that interferes with effective drug delivery. Moreover, due to their insolubility, sparingly soluble or almost insoluble drugs cannot readily be delivered by either solution-diffusion or membrane-controlled delivery systems.

### SUMMARY

There remains a need and opportunity for an improved formulation that improves the bioavailability and release rate of Valsartan.

Thus, to enable improved therapy in cases where a drug has poor solubility and consequently, poor bioavailability, a gastroretentive pulsatile pharmaceutical delivery system is herein disclosed. After oral administration, such gastroretentive dosage form will remain in the stomach and release the drug in a controlled and prolonged manner. Examples of gastroretentive dosage forms are floating dosage forms and dosage forms that expand, swell or unfold in the stomach.

The present invention provides pharmaceutical delivery systems that improve the bioavailability of Valsartan wherein the medicament has an improved solubility, improved residence time and improved release profile such that the drug or active agent is released from the delivery system at multiple times. According to an aspect of the invention, a pharmaceutical delivery system for the oral delivery of Valsartan is provided, comprising a Valsartan-containing delivery system that is pulsatile, gastroretentive and wherein Valsartan is treated with solubility enhancers and/or permeation enhancers.

In further embodiments, the pharmaceutical delivery system comprises an immediate release (IR) component, a modified release (MR) component and Valsartan. In certain embodiments, Valsartan is independently incorporated into the individual components of the system, such as the IR and/or MR components. Valsartan can be in its unenchanced form, treated with a permeation enhancer, treated with a solubility enhancer, or any combination thereof.

In certain embodiments, the MR component comprises a single first delayed release region. In further embodiments, the MR component is multi-regioned and comprises a first delayed release region and a second delayed release region. In yet another embodiment, the multi-regioned MR component comprises three or more delayed release regions.

In certain embodiments, the pharmaceutical delivery system is pulsatile such that upon a single administration of the system, multiple dosages of Valsartan are subsequently and sequentially released from the system. Each dose corresponds to individual pulses of Valsartan that are released from the system at different times. In certain embodiments, the first pulse is released from the IR component and a second pulse is released from the first delayed release region at some time subsequent to the first pulse. In other embodiments, a first pulse is released from the IR component, a second pulse is released from the second delayed release region, and a third pulse is released from the first delayed release region. Valsartan can be in its unenchanced form, treated with a permeation enhancer, treated with a solubility enhancer, or any combination thereof.

In certain embodiments, the MR component comprises a swellable gelled-matrix such that the system swells, expands, floats, adheres to the gastrointestinal mucosal lining or any combination thereof. The swellable gelled-matrix can swell, expand or unfold when in the presence of a liquid such as the gastric milieu of the gastrointestinal tract. The swellable gelled-matrix allows prolonged residence time in the gastrointestinal tract by maintaining the system in a gastroretentive manner. The system thereby delivers the therapeutically effective dosages of Valsartan before the system is moved to the small intestines. The system is retained in the gastrointestinal tract such that all pulses of Valsartan are delivered before the system is delivered to the small intestines. In some embodiments, the delivery system can be adapted to deliver one or more pulses in the small intestines. In further embodiments, the MR component is multi-regioned and comprises multiple swellable gelled-matrixes.

In certain embodiments, the IR component and the delayed release region(s) of the MR component are in axial or layered communication with each other.

In some embodiments, the system is a tablet, capsule, granule, bead, a gel, a liquid or combination thereof. In certain other embodiments, the system comprises a compressed powder and polymeric materials. Valsartan can be incorporated in the MR component as granules, compressed powder or any combination thereof. In certain other embodiments, Valsartan is entrapped between the regions of the MR component or between the MR and the IR components. In certain embodiments, Valsartan is encircled by the first delayed release region. In certain other embodiments, Valsartan is infused into the swellable gelled matrix of the first delayed release region, the second delayed release region or both. In certain other embodiments the IR component comprises a polymeric material infused with Valsartan. In further embodiments the delivery system comprises a modified release component which comprises a first delayed release region and a second delayed release region. The first and second delayed release regions independently comprise a compressed powder layer, a polymeric layer, a swellable gelled matrix or a combination thereof.

In other aspects of the invention, methods of treatment comprising administering the above described delivery system are also disclosed herein. In certain embodiments, the gastroretentive pulsatile pharmaceutical delivery systems herein described, are used in treating subjects in need thereof. In further embodiments the delivery systems are used to treat subjects suffering from high blood pressure, congestive heart failure or post myocardial infarction. In some embodiments the delivery system is administered concomitantly or sequentially with an effective amount of a second active agent capable of delaying gastric emptying.

### DETAILED DESCRIPTION

### Definitions

The term “pulsatile,” “pulsatile dosage form,” or “pulsatile delivery,” as used herein, is intended to represent a device that has the ability to release multiple doses upon a single administration of the device to a subject. The individual doses can be administered at a variety of intervals, depending on the formulation of the pulsatile pharmaceutical delivery system or gastroretentive pulsatile pharmaceutical delivery system, as described herein.

The term “gastroretentive,” as used herein, is intended to represent the ability of the pharmaceutical delivery system of the invention to remain within the gastrointestinal tract while delivering a therapeutic agent (e.g., Valsartan). As used herein, “gastroretentive” also refers to the ability of the pharmaceutical delivery system of the invention to insulate a therapeutic agent (e.g., Valsartan) from the gastric environment that would otherwise degrade the therapeutic agent or remove the therapeutic agent from the gastric environment (e.g., gastric emptying). As such, the components of the gastroretentive, pharmaceutical delivery system of the invention allow a therapeutic agent (e.g., Valsartan) to exist in the gastric environment for extended time periods (compared to the ability of the therapeutic agent to exist in the gastric environment without the aid of the components of the invention). By allowing the therapeutic agent to exist in the gastric environment for extended time periods, the therapeutic agent (e.g., Valsartan) can be delivered to a subject at a controlled rate over a period of time.

The term “gastroretentive manner,” as used herein, includes the ability of the system to reside in the gastrointestinal tract beyond one period of gastric emptying.

The term “incorporated,” “incorporated within,” or “incorporating” as used herein, is intended to represent embodiments wherein the drug can be entrapped, infused or encircled by one or more of the immediate release components, or any number of the delayed release regions. The term “entrapped” is intended to represent embodiments wherein the active agent is sandwiched between two components, such as between the IR component and the MR component. The term “infused”is intended to represent embodiments wherein the active agent is dispersed or distributed throughout a polymeric layer.

The term “pulse,” as used herein, is intended to represent each individual temporal release of the active agent from the device to the surrounding environment. For example, a first pulse can occur substantially immediately upon oral administration of the delivery device such that the plasma concentration of the active agent is peaked. A second pulse can occur at some time after the first pulse (e.g., 3 to 14 hours after the first pulse). The second pulse can be followed by third pulse, fourth pulse, fifth pulses, etc.

The term “immediate release component,” “IR.” or “IR component,” as used herein, is intended to represent regions of the device from where the drug is released substantially immediately upon oral administration to provide a first pulse.

The term “modified release component,” “MR,” or “MR component,” as used herein, is intended to represent one or more (multi-regioned) delayed release regions that are in axial or layered communication with each other and with the immediate release component. The “modified release component,” “MR,” or “MR component” can be adapted to provide a second pulse, or a second pulse and a third pulse of the agent from the delivery system. In a particular embodiment, the MR component comprises one or more delayed release (DR) regions.

The term “delayed release regions,” as used herein, is intended to represent embodiments of the modified release component regions that can be in axial or layered communication with each other or with the immediate release component. The delayed release regions retard the release of Valsartan such that it is released at some time subsequent to the release from the immediate release component. As described herein, the release rate of Valsartan from the delayed release region is controlled by changing its formulation parameters.

The term “sustained delivery” is used to refer to a drug formulation that provides for gradual release of a drug over an extended period of time, and that preferably, although not necessarily, results in substantially constant blood levels of a drug over an extended time period such as up to about 72 hours, about 66 hours, about 60 hours, about 54 hours, about 48 hours, about 42 hours, about 36 hours, about 30 hours, about 24 hours, about 18 hours, about 12 hours, about 10 hours, about 8 hours, about 7 hours, about 6 hours, about 5 hours, about 4 hours, about 3 hours, about 2 hours, or about 1 hour after drug administration.

The term “delivery system” or “delivery device” denotes generically a means or system for storing and subsequently delivering or releasing a beneficial ingredient or agent or mixture thereof.

The term “multi-regioned,” as used herein, is intended to describe embodiments where a component of the system comprises more than one region. For example, the modified release component, in its multi-regioned embodiment can comprise two or more delayed release regions.

The term “axial communication,” as used herein, is intended to describe embodiments where the layers or regions of the device of the invention are spherical, elliptical, curved or otherwise do not have a terminal surface area.

The term “layered communication” is intended to describe embodiments where the layers or regions are stacked, as in a laminate, where the layers can be co-terminus or can have different lengths and/or widths. Layers, regions or components in layered communication will have terminal surface area, though the surface area can not necessarily be co-terminus.

The term “release controlling materials” is intended to embody materials that modify Valsartan's time of release from the device. Such materials can be chosen from the list herein described.

The term “release modifying ingredients” is intended to embody ingredients that modify Valsartan's rate of absorption once released from the device. Such ingredients can be selected from the list herein described.

The term “encircled,” as used herein, is intended to represent an embodiment wherein a layer or region is spherically or elliptically surrounded. For example, as described herein, an MR component of the pharmaceutical composition can be encircled by an IR component.

The term “infused,” as used herein, is intended to represent embodiments wherein the drug is distributed throughout a polymeric layer.

The term “entrapped.” as used herein, is intended to represent embodiments wherein the drug is situated between two regions or layers.

The term “core,” as used herein, is intended to represent the centermost region of a spherical, elliptical or otherwise round embodiment of the pharmaceutical delivery system. For example, the core of the system can be a powder, pressed powder, liquid, gel, or any other form situated in the innermost region of the system.

The term “capsule” refers to a special container or enclosure made of methylcellulose, polyvinyl alcohols, or denatured gelatins or starch for holding or containing compositions comprising the active ingredients. Hard shell capsules are typically made of blends of relatively high gel strength bone and pork skin gelatins. The capsule itself can contain small amounts of dyes, opaquing agents, plasticizers and preservatives.

The term “tablet” refers to a compressed or molded solid dosage form containing the active ingredients with suitable diluents. The tablet can be prepared by compression of mixtures or granulations obtained by wet granulation, dry granulation or by compaction.

The term “solubility enhanced” refers to an active agent whose rate of solubility or degree of solubility is improved by means of a chemical compound that, when present in solution in a solvent, increases the solubility of the active agent in the solvent, but which chemical compound is not itself a solvent for the active agent.

The term “swellable gelled matrix,” as used herein, refers to a polymeric hydrogel capable of expanding upon contact with a liquid environment.

The term “period of gastric emptying” refers to the time between ingestion of an agent and the time necessary for the ingested agent to be removed to the small intestines.

The term “subject” is intended to include animals, which are capable of suffering from or afflicted with conditions that can be treated with Valsartan, such as high blood pressure, congestive heart failure, or post-myocardial infarction. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a disease or disorder that can be treated with Valsartan.

### Pulsatile Dosage Form

The advantages of using controlled drug delivery systems are many. Of major importance in controlled drug therapy is the improved efficiency in treatment. Controlled drug therapy reduces the required frequency of administration, and single doses at periodic intervals are sufficient, resulting in improved patient compliance.

The present invention provides pharmaceutical delivery systems that improve the bioavailability of Valsartan by providing a gastroretentive system that is retained in the gastrointestinal tract in a gastroretentive manner while simultaneously providing a pulsatile delivery of Valsartan for a sustained, controlled, release. According to one aspect of the invention, the pharmaceutical carrier device is a pulsatile dosage form wherein Valsartan is treated with solubility enhancers, permeation enhancers, lipid carriers or combinations thereof. In further aspects of the invention, the pharmaceutical carrier device is a pulsatile dosage form wherein Valsartan is treated with solubility enhancers, permeation enhancers, lipid carriers or combinations thereof, and the device is gastroretentive.

The present invention provides a pulsatile pharmaceutical delivery system for the delivery of Valsartan. Valsartan can be in an enhanced form, meaning it can be treated with a permeation enhancer or a solubility enhancer as discussed below. The present invention further provides pharmaceutical delivery systems adapted to provide a therapeutically effective blood concentration level of enhanced or unenchanced Valsartan. According to the present invention, there are provided pharmaceutical delivery systems adapted to provide therapeutically effective blood concentration levels of Valsartan for a sustained period of time for up to about twenty-four hours based on a single oral administration of the delivery system.

In one embodiment, the delivery system comprises an immediate release (IR) component that provides a first pulse of Valsartan. The system further comprises a modified release (MR) component that provides at least one additional pulse of Valsartan. Valsartan is incorporated into at least one of the IR or MR components. Valsartan incorporated therein can be in an enhanced or unenchanced form. The IR and MR components are in axial or layered communication with each other and the delivery system delivers Valsartan in a therapeutically effective manner.

Valsartan, in its unenhanced or enhanced form, is incorporated into a pulsatile pharmaceutical delivery system wherein there is an immediate release (IR) component, and a modified release (MR) component. The IR and the MR components can individually contain Valsartan in the enhanced or unenhanced form, or in any combination thereof. For example, the IR component can contain unenhanced or enhanced Valsartan, or both. The MR component can simultaneously contain Valsartan in its enhanced form, unenhanced form or both. The IR component causes Valsartan to be released substantially immediately and substantially completely upon oral administration. In certain embodiments the MR component is multi-regioned and comprises at least two delayed release (DR) regions that cause the delayed release of Valsartan. The DR region causes Valsartan, incorporated therein, to be released at some time subsequent to the release of Valsartan in the IR component. The DR regions determine the time of release and the time of release is dependent upon the materials used to retard or control the release profile of Valsartan.

The materials used to retard or control the release profile of Valsartan are herein defined to be release controlling materials. These release controlling materials can be selected from cellulose and cellulose derivatives such as methylcellulose, ethyl cellulose, hydroxypropyl methylcellulose, hydroxypropyl cellulose, cellulose acetate phthalate, cellulose acetate, cellulose acetate butyrate, cellulose acetate propionate, cellulose acetate trimellitate, cellulose carboxymethyl ethers and their salts, hydroxypropyl methylcellulose phthalate, hydroxypropyl methylcellulose acetate succinate, polyethylene, polyquaternium-1, polyvinyl acetate (homopolymer), polyvinyl acetate phthalate, propylene glycol alginate, polyvinyl methacrylate(PVM)/methacrylic acid(MA) copolymer, polyvinyl pyrrolidone (PVP), PVP/dimethiconylacrylate/polycarbamyl/polyglycol ester, PVP/dimethylaminoethyl methacrylate copolymer, PVP/dimethylaminoethylmethacrylate/polycarbamyl/polyglycol ester, PVP/polycarbamyl polyglycol ester, PVP/vinyl acetate (VA) copolymer, lanolin and lanolin derivatives, glyceryl monostearate, stearic acid, paraffins, beeswax, carnauba wax, tribehenin, polyalkylene polyols like polyethylene glycols, gelatin and gelatin derivatives, alginates, carbomers, polycarbophils, methacrylic acid polymers and copolymers, carrageenans, pectins, chitosans, cyclodextrins, lecithins, natural and synthetic gums containing galactomannans like xanthan gum, tragacanth, acacia, agar, guar gum, karaya gum, locust bean gum, gum arabic, and the like, used either alone or in combination.

The release controlling materials can also be enteric polymers. Suitable enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methamethacry ate copolymers and shellac. These polymers can be used as a dry powder or an aqueous dispersion. Some commercially available materials that can be used are methacrylic acid copolymers sold under the trademark Eudragit (L100, S100, L30D) manufactured by Rhom Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

The release controlling materials can also be water insoluble. Suitable water insoluble polymers useful in the invention include cellulose derivatives (e.g. ethylcellulose), polyvinyl acetate (Kollicoat SR30D Z from BASF), neutral copolymers based on ethyl acrylate and methylmethacrylate, copolymers of acrylic and methacrylic acid esters with quaternary ammonium groups, such as Eudragit NE, RS or RS30D, RL or RL30D and the like.

The IR and MR components are formulated such that a pulsatile release of Valsartan is achieved. In certain embodiments, the IR component is in axial or layered-communication with the MR component such that the IR component encompasses the MR component. The terms “encompass,” “encompassing,” “encompassed,” or any other similar permutations are intended to mean that the layers are in axial or layered-communication. The term axial communication means that the layers or regions are spherical, elliptical, semi-circular or any other embodiment wherein the layers are curved about each other. The term layered communication means that the layers or regions are planar and definite such as where the layers are stacked upon each other and have a terminal surface area. The layers can be co-terminus or can be uneven in width, length or both.

In certain embodiments, the delivery system comprises an IR component and a multi-regioned MR component with at least two delayed release (DR) regions. In a more preferred embodiment, the MR component comprises a first delayed release region that is encompassed by a second delayed release region that is itself encompassed by the IR component. The second delayed release region is disposed between the first delayed release region and the IR component. The IR component, the first DR region and the second DR region are in axial or layered communication. In certain embodiments, a first pulse of Valsartan, enhanced or unenhanced, is released from the IR component and a second pulse is released upon the disintegration, unfolding or swelling of a swellable gelled-matrix of the first delayed release region. In further embodiments, a first pulse is released from the IR component, a second pulse is released upon the disintegration, unfolding or swelling of a swellable gelled-matrix of the second delayed release region and a third pulse is released upon the disintegrations, unfolding or swelling of a swellable gelled-matrix of the first delayed release region. As used herein, the term “disintegrate” is meant to represent the breaking apart, dissolution or erosion of the polymeric material used to form the DR regions.

In certain other aspects of the invention, Valsartan, in its enhanced or unenhanced form, is incorporated into a pulsatile pharmaceutical delivery system wherein there is an IR component and multiple MR components. The IR and the MR components can individually contain Valsartan in the enhanced or unenhanced form, or in any combination thereof. The MR component can simultaneously contain Valsartan in its enhanced form or unenhanced form, or both. The IR component causes Valsartan, enhanced or unenhanced, to be released substantially immediately and substantially completely upon oral administration. The MR component comprises multiple delayed release (DR) regions that allow for the delayed release of Valsartan. The MR component can comprise two or more DR regions. The DR regions allow Valsartan, incorporated individually therein, to be released at some time subsequent to the release of Valsartan in the IR component. The DR regions can be released simultaneously, or sequentially, relative to each other. Whether the DR regions are released simultaneously or sequentially depend upon the materials chosen to retard the release profile of Valsartan therein incorporated. The IR and MR components are formulated such that a pulsatile release of Valsartan is achieved.

In certain embodiments, the pulsatile pharmaceutical delivery systems are formulated as closed capsules or as multi-layered devices wherein the IR and MR components generate at least two release profiles of Valsartan upon administration. Each capsule or multi-layered device can further comprise a compressed tablet or plurality of compressed tablets or plurality of beads, plurality of granules, or plurality of particles or combinations thereof situated within the capsule. The IR component releases Valsartan substantially immediately following oral administration to provide an immediate release of an initial dose. The MR component further comprises a DR region. The DR region consists of a plurality of beads, plurality of granules or plurality of particles or combinations thereof, that releases Valsartan at about 3 to about 14 hours following oral administration to provide a second dose. Where the MR component comprises multiple DR regions, a second DR region can release Valsartan at about 3 to about 14 hours following oral administration to provide a second dose while a first DR region releases Valsartan at about 14 hours to about 18 hours following oral administration to provide a third dose.

In certain other embodiments, a tablet formulation of the pulsatile pharmaceutical delivery system is disclosed. The tablet has an IR component and a MR component. The IR component contains Valsartan and provides an immediate release of an initial dose. The MR component can be encompassed by the IR component. The MR component can be incorporated with Valsartan and comprises one or more DR regions. For example, the MR component can comprise a second DR region and a first DR region encompassed by the second DR region. The first and second DR regions release Valsartan at some time subsequent to the release of Valsartan in from the IR component. Preferably, the second DR region releases Valsartan at about 3 hours to about 14 hours following oral administration while the first DR region releases Valsartan at about 14 hours to about 18 hours following oral administration. In certain embodiments of this aspect, the IR component is in axial or layered communication with the MR component. The first DR region can be encompassed by the second DR region. The first DR region can be in axial or layered communication with the second DR region. The second DR region comprises an inner layer that is incorporated with Valsartan. The first DR region comprises a core layer that is incorporated with Valsartan.

In another embodiment, the pulsatile pharmaceutical delivery system is formulated such that the first DR region is comprised of beads or granules that release Valsartan upon erosions, disintegration, swelling, or unfolding of the first DR region. The IR component is incorporated with Valsartan and provides an immediate release of an initial dose. The IR component is in axial or layered communication with the MR component and is encompassed by the IR component. The MR component comprises one or more DR regions that are incorporated with Valsartan. For example, the MR component can comprise a first DR region and optionally a second DR region encompassing the first DR region. The first and second DR regions release Valsartan at some time subsequent to the release of Valsartan from the IR component. Preferably, the second DR region releases Valsartan at about 3 hours to about 14 hours following oral administration. The first DR region releases Valsartan at some time subsequent to the release of Valsartan from the second DR region. Preferably, the first DR region releases Valsartan at about 14 hours to about 18 hours following oral administration. Accordingly, in some embodiments, the IR component comprises an outer layer wherein Valsartan is incorporated therein for immediate release of an initial dose. An inner layer borders the IR component from the second DR region. The inner layer is incorporated with Valsartan. The first DR region comprises a core of beads or granules.

In certain other embodiments, the pulsatile pharmaceutical delivery system is formulated as a tablet such that the second DR region comprises a polymeric layer that is infused with Valsartan. The IR component contains Valsartan and provides an immediate release of an initial dose. The MR component is encompassed by the IR component and is in axial or layered communication with the IR component. The MR component comprises one or more DR regions that are incorporated with Valsartan. For example, the MR component can comprise a first DR region and an optional second DR region encompassing the first DR region. The first and second DR regions release Valsartan at some time subsequent to the release of Valsartan in from the IR component. Preferably, the second DR region releases Valsartan at about 3 hours to about 14 hours following oral administration. The first DR region releases Valsartan at some time subsequent to the release of Valsartan from the second DR region. Preferably, the first DR region releases Valsartan at about 14 hours to about 18 hours following oral administration. In some embodiments, the first DR region comprises Valsartan as a liquid, beads or granules. Accordingly, in some embodiments, the IR component comprises an outer layer wherein Valsartan is incorporated therein for immediate release of an initial dose. An inner layer borders the IR component from the second DR region. The second DR region comprises a polymeric layer incorporated with Valsartan where Valsartan is infused into the layer. The first DR region is encompassed by the inner layer and has a core of Valsartan encompassed by the inner layer. The core of Valsartan can be in the form of a liquid, beads or granules.

In certain other embodiments, the pulsatile pharmaceutical delivery system is formulated such that Valsartan is incorporated into the second DR region as a liquid or gel. The IR component is incorporated with Valsartan and provides an immediate release of an initial dose. In certain preferred embodiments, when Valsartan is incorporated in the IR component, it is in the form of a pressed powder infused with Valsartan. In certain other embodiments, when Valsartan is incorporated in the IR component, it is infused into a polymeric material capable of biodegradation, disintegration or expansion. In certain other embodiments, when Valsartan is incorporated into the IR region, it is entrapped between a polymeric material used to form the IR component and the polymeric material of the MR component. The MR component is encompassed by the IR component and is in axial or layered communication with the IR component. The MR component comprises one or more DR regions that are incorporated with Valsartan. In certain embodiments, the IR component is an outer layer wherein Valsartan is incorporated. The IR component provides an immediate release of an initial dose of Valsartan. The MR component is encompassed by the IR component and is in axial or layered communication with the IR component. The MR component comprises one or more DR regions that are incorporated with Valsartan. An inner layer can border the IR component and the MR component. In certain embodiments the inner layer does not contain Valsartan. A second DR region can comprise Valsartan incorporated therein in liquid or gel form. A first DR region can comprise beads and granules distributed throughout the Valsartan liquid or gel of the second DR region. The layers of the tablet comprise an outer layer, inner layer and an admixture of the DR regions. Upon disintegration of the inner layer, the first DR region releases Valsartan immediately. The beads and granules release Valsartan at some time subsequent to the first DR region.

In certain embodiments, the first DR region of the system comprises a polymeric material as discussed below, wherein Valsartan is encircled by the polymeric material. The encircled Valsartan can be in the form of a pressed powder, granules, beads, a gel, a liquid or any combination thereof. The second DR region of the system comprises Valsartan infused into the polymeric material of the second DR region or is entrapped between the polymeric material of the second DR region and the polymeric material of the first DR region. The IR component comprises a pressed powder infused with Valsartan or comprises a polymeric material infused with Valsartan. In certain other embodiments the IR component comprises Valsartan entrapped between the polymeric material of the IR region and the polymeric material, as discussed below, of the DR regions.

### Gastroretentive Dosage Form

The present invention further provides a gastroretentive form of the pulsatile pharmaceutical delivery system as described above wherein the delivery system is also is retained in the gastrointestinal tract beyond at least one period of gastric emptying. Typically, a drug is absorbed most efficiently through the stomach and the proximal part of the small intestine. Gastroretentive (GR) controlled delivery systems of the invention can be advantageous in the administration of drugs having an otherwise narrow absorption window. These drugs are usually absorbed in limited segments of the upper parts of the gastrointestinal tract (most often in the duodenum and jejunum). In addition, many of these drugs are absorbed by active transport systems in the aforementioned upper parts of the gastrointestinal tract, or are poorly soluble at intestinal medium pH. By prolonging the duodenal delivery of drugs having a narrow absorption window, their bioavailability and their therapeutic effect can be enhanced.

The delivery system of the invention is so designed as to allow for its disintegration after the desired drug-release time, so that all of its components are evacuated from the stomach. Accordingly, a pharmaceutical delivery system that is capable of a sustained residence time in the GI would be beneficial especially where a pulsatile pharmaceutical delivery system is used to administer multiple dosages from a single administration of the pulsatile pharmaceutical delivery system.

In certain aspects, the present invention therefore relates to a gastroretentive pulsatile pharmaceutical delivery system for the controlled release of Valsartan in the gastrointestinal tract. In certain aspects, the system comprises a pulsatile dosage form, as described above, having (a) an immediate release (IR) component (b) a modified release (MR) component and (c) Valsartan in its enhanced or unenchanced form incorporated in the IR component, the MR component, or both, and (d) one or swellable gelled-matrixes capable of expanding, swelling, disintegrating, adhering to the gastrointestinal mucosa or floating. The swellable gelled-matrix of the MR component expands or swells upon contact with the gastrointestinal milieu such that the surface area of the system is broader the diameter of the pyloric sphincter. The swellable gelled-matrix is distributed such that the matrix is in axial or layered communication with the IR component, the MR component, or both. In certain embodiments, the swellable gelled-matrix is infused with Valsartan in its enhanced or unenchanced form. In certain other embodiments the swellable gelled-matrix of the first DR region entraps Valsartan in its enhanced or unenchanced form and the swellable gelled matrix of the second DR region entraps Valsartan between the polymeric material of the first DR region and the polymeric material of the second DR region. Where the MR component comprises multiple delayed release (DR) regions, the matrix can also be in axial or layered communication with one of more of the DR regions.

Both enteric and water insoluble polymers can be used in forming the matrix. These polymers can be plasticized. Representative examples of plasticizers that can be used to plasticize the matrixes include triacetin, tributyl citrate, triethyl citrate, acetyl tri-n butyl citrate diethyl phthalate, castor oil, dibutyl sebacate, acetylated mono glycerides and the like or mixtures thereof. The plasticizer can be about 3 to 30 wt. % and more typically about 10 to 25 wt. % based on the polymer. The type of plasticizer and its content depends on the polymer or polymers, and/or the nature of the coating system (e.g., aqueous or solvent based, solution or dispersion based and the total solids).

The matrix will control the gastroretentivity of the system by maintaining the system in its desired configuration for a predetermined time. Evacuation of the system from the stomach should take place after the matrix undergoes biodegradation, bioerosion, dissolution or disintegration, thus enabling separation of the matrix to its smaller fragments or collapse of the matrix and inevitably the system in any other way.

Gastroretentive characteristics can be incorporated into dosage forms/drug delivery systems by techniques such as treating the active agent (e.g., Valsartan) with polymers having specific affinity to bind with gastric mucosa, reducing specific gravity of the dosage form leading to floatation, increasing size of the dosage form such that it is greater than the pyloric diameter, and/or using chemicals which delay gastric emptying, and the like, or a combination of more than one such techniques. In an embodiment, the gastroretentivity of the dosage form composition can also be achieved by delaying the gastric emptying time such as by administration of food. In certain further embodiments, a second active agent, capable of delaying gastric emptying, can be administered concomitantly or sequentially with the pharmaceutical delivery system.

In certain other aspects of the invention, the gastroretentivity is achieved by reducing the specific gravity of the delivery system such that the delivery system floats in the gastric milieu. Particular substances used to reduce the specific gravity such that the system can float in the gastric milieu, include, but are not limited to, gas generating agents such as water soluble carbonates, sulfites and bicarbonates, such as sodium carbonate, sodium bicarbonate, sodium metabisulfite, calcium carbonate, and their mixtures. While not wishing to be bound by any specific theory, it is believed that as the acidic environment of the stomach enters in the gelled matrix, it reacts with the gas liberating agent to liberate gas. The liberated gas gets entrapped in the gel matrix and releases slowly as the drug is diffused or delivered from the gel.

In certain other aspects of the invention, the gastroretentivity is achieved by incorporating a matrix into the pulsatile pharmaceutical delivery system such that it acts to expand the size of the system such that it is greater than the diameter of the pyloric sphincter. The matrix can be in axial or layered communication with an immediate release component and a modified release component, such as described above. The drug, such as Valsartan, can also be incorporated into the matrix, the immediate release component, the modified release component, or any combination thereof. The matrix will release Valsartan at some time subsequent to the release of Valsartan from the immediate release component.

In certain embodiments, the DR regions of the MR component comprise a swellable gelled-matrix. The matrix can absorb the gastric fluid and swell as a result of the absorbed fluid. Emptying of the device into the plyorous is delayed by having a swellable gelled-matrix component which is expandable or swellable upon contact with gastric juice or gastric milieu. In a particular embodiment, the swellable gelled matrix is a polymeric hydrogel. In some aspects, the swellable gelled-matrix also acts to delay release of Valsartan until the swellable gelled-matrix is degraded.

As used herein, the term “polymeric hydrogel” refers to a class of polymeric materials that are extensively swollen in an aqueous medium, but do not dissolve in water. In general terms, hydrogels are prepared by polymerization of a hydrophilic monomer under conditions where the polymer becomes cross-linked in a three dimensional matrix sufficient to gel the solution. Bioartificial or semi-synthetic hydrogels can also be prepared by the covalent addition of the hydrophilic polymer to the surface of a protein an that the polymer and protein form a further covalently cross-linked three dimensional matrix. This class of hydrogels, made from a synthetic polymer and a biopolymer, has been recently reviewed in Giusti, P. et al., Trends in Polymeric Science, (261-267, 1993), incorporated herein by reference.

Matrices that can be used in the pulsatile pharmaceutical delivery system of the invention can also include swellable hydrogels containing binders that are water-swellable polymers, and suitable polymers are those that are non-toxic, that swell in a dimensionally unrestricted manner upon imbibition of water, and that release the drug gradually over time. Examples of polymers meeting this description are: cellulose polymers and their derivatives including, but not limited to, hydroxymethyl cellulose, hydroxyethyl cellulose, hydroxypropyl cellulose, hydroxypropylmethyl cellulose, carboxymethylcellulose, and microcrystalline cellulose polysaccharides and their derivatives polyalkylene oxides polyethylene glycols chitosan poly(vinyl alcohol) xanthan gum maleic anhydride copolymers poly(vinyl pyrrolidone) starch and starch-based polymers maltodextrins poly(2-ethyl-2-oxazoline) poly(ethyleneimine) polyurethane hydrogels crosslinked polyacrylic acids and their derivatives.

Where a swellable gelled-matrix is incorporated, entry of the system into the gastric milieu will cause the system to expand following contact with the gastric milieu. In other embodiments, the polymeric hydrogel is chosen such that the system is mucoadhesive such that the gelled-matrix can adhere to the gastrointestinal mucosa. In other embodiments, the matrix comprises a hydrogel and one or more other polymers that are mucoadhesive. In other embodiments, the polymeric hydrogel is chosen such that the system can float in the gastric milieu. In other embodiments, the matrix comprises a hydrogel and one or more other polymers that reduces the specific gravity of the system such that system floats in the gastric milieu.

In certain embodiments, the gelled-matrix, upon expansion, causes the gastroretentive pulsatile pharmaceutical delivery system to be retained in the stomach beyond the normal time for gastric emptying, thus preventing evacuation from the stomach before the drug is released. In further preferred embodiments the system is retained in the stomach beyond at least one period of gastric emptying. In further embodiments the system is retained in the stomach for at least four hours.

The device can contain an active compound (e.g., Valsartan) which is mainly released into the gastric juice in a controlled manner by the incorporated pharmaceutical form. The pharmaceutical form can be, for example, a pulsatile pharmaceutical delivery system as described above. In certain embodiments, the device according to the invention can be easily rolled or folded and can be filled into capsules.

In a further illustrative embodiment, a solid pharmaceutical composition in the form of an expanding multilayer or multi-component system for oral administration is adapted to deliver Valsartan from a first layer or first component immediately upon oral administration or immediately upon reaching the gastrointestinal tract, and to deliver a second pharmaceutical agent, which can be Valsartan or a different agent, in a controlled manner over a specific time period. The second layer or component is also adapted to provide an expanding nature for the dosage system, thereby making the dosage system have greater retention in the stomach.

According to one aspect of the present invention, a pulsatile pharmaceutical delivery system as described above is treated with polymers having specific affinity to bind with gastric mucosa such that residence time in the upper GI is increased. In other aspects of the present invention, the pulsatile pharmaceutical delivery system described above is formulated such that the specific gravity of the dosage form leads to floatation in the gastric milieu. In other aspects of the present invention, the pulsatile pharmaceutical delivery system described above is formulated such that its size is larger than the diameter of the pyloric of a mammal in need thereof.

The gastroretentive pulsatile pharmaceutical delivery system can be in the form of raw powder, or soluted, dispersed or embedded in a suitable liquid, semisolid, micro- or nanoparticles, micro- or nanospheres, tablet, capsule or a suitable matrix. The drug, or mixtures of drugs, in any of said forms, can be embedded in at least one layer of the delivery system of the invention. Alternatively, the drug can be entrapped between any of the layers defining the IR component, the MR component or the multiple DR regions. For example, where the device has layered communication between the components and/or regions, a semi-solid drug can be contained between any two layers of the matrix. Another example is that in which the drug is contained in a tablet, a capsule or any pharmaceutically compatible matrix, and the drug-containing tablet, capsule or pharmaceutically compatible matrix are entrapped between any two layers of the matrix. Such multi-layered embodiments preferably have a shielding layer. Alternatively, the drug, preferably contained with said drug-containing means, can be tethered by tethering means, or otherwise attached, to the delivery system of the invention.

### Solubility and Permeation Enhanced Valsartan

In any of the embodiments described herein, Valsartan can be modified. Valsartan can be treated with release modifying ingredients to form a composition. Such release modifying ingredients are selected from, but are not limited to, the group comprising wetting agents, solubilizers, surfactants, plasticizers, solvents, pH modifiers, tonicity adjusting agents, and the like or mixtures thereof. Suitable examples of such ingredients include reaction products of natural and hydrogenated vegetable oils and ethylene glycol, e.g., polyoxyethylene glycolated natural or hydrogenated castor oil such as CREMOPHOR. Other suitable products include polyoxyethylene sorbitan fatty acid esters. e.g., TWEEN, polyoxyethylene fatty acid esters, e.g., MYRJ and CETIOL HE, polyoxyethylene polyoxypropylene copolymers, e.g., PLURONIC and polyoxyethylene polyoxypropylene block copolymers, e.g., POLOXAMER, dioctylsodiumsulfosuccinate, sodium lauryl sulphate, propylene glycol mono-and di-fatty acid esters, e.g., MIGLYOL 840, bile salts such as alkali metals salts, e.g., sodium taurocholate, polyethylene glycols, propylene glycol, triacetin, diacetin, diethyl phthalate, dibutyl phthalate, castor oil, triethyl citrate dibutyl sebacate, sodium chloride, potassium chloride, lactose, mannitol, sucrose, sorbitol, sodium hydroxide, potassium hydroxide, sodium bicarbonate, sodium citrate, citric acid, hydrochloric acid, lactic acid, tartaric acid, malic acid, and the like or mixtures thereof.

Valsartan can be treated with surfactants to form a Valsartan composition. The surfactants can include polyethylene glycol sorbitan fatty acid esters, polyethylene glycol fatty acid monoesters, PEG-fatty acid diesters, hydrophilic trans-esterification products of alcohols or polyols with at least one member of the group consisting of natural and/or hydrogenated oils. The most commonly used oils are castor oil or hydrogenated castor oil, or an edible vegetable oil such as corn oil, olive oil, peanut oil, palm kernel oil, almond oil. Preferred polyols include glycerol, propylene glycol, ethylene glycol, polyethylene glycol, sorbitol and pentaerythritol. Preferred hydrophilic surfactants in this class include PEG-35 castor oil, polyoxyethylene-polypropylene copolymer (Lutrol, BASF), and PEG-40 hydrogenated castor oil.

Valsartan can also be treated with permeation enhancers to form a Valsartan composition. Such permeation enhancers can be selected from, but are not limited to, the group comprising Vitamin E tocopheryl propylene glycol succinate, piperine, a lipid, or a surfactant, or mixtures thereof.

Valsartan can be treated with solubility enhancers or solubility enhancing ingredients to form a Valsartan composition. The solubility enhancing agents can be selected from, but are not limited to, the group comprising PEG-20-glyceryl, stearate (Capmul® by Abitec), PEG-40 hydrogenated castor oil (Cremophor RH 40® by BASF). PEG 6 corn oil (Labrafil® by Gattefosse), lauryl macrogol-32 glyceride (Gelucire 44/14® by Gattefosse), stearoyl macrogol glyceride (Gelucire 50/13® by Gattefosse), polyglyceryl-10 mono dioleate (Caprol® PEG 860 by Abitec), propylene glycol oleate (Lutrol OP® by BASF), propylene glycol dioctanoate (Captex® by Abitec), propylene glycol caprylate/caprate (Labrafac® by Gattefosse), glyceryl monooleate (Peceol® by Gattefosse), glycerol monolinoleate (Maisine® by (Jattefosse), glycerol monostearate (Capmul® b Abitec), PEG-20 sorbitan monolaurate (Tween 20® by ICI), PEG-4 lauryl ether (Brij 30® by ICI), sucrose distearate (Sucroester 7® by Gattefosse), sucrose monopalmitate (Sucroester 15® by Gattefosse), polyoxyethylene-polyoxypropylene block copolymer (Lutrol® series BASF), polyethylene glycol 660 hydroxystearate, (Solutol® by BASF), sodium lauryl sulphate, sodium dodecyl sulphate, dioctyl suphosuccinate, L-hydroxypropyl cellulose, hydroxylethylcellulose, hydroxy propylcellulose, propylene glycol alginate, sodium taurocholate, sodium glycocholate, sodium deoxycholate, betains, polyethylene glycol (Carbowax® by DOW), d-α-tocopheryl polyethylene glycol 1000 succinate (Vitamin E TPGS® by Eastman), or mixtures thereof.

More preferably, the solubility enhancing ingredients can be PEG-40 hydrogenated castor oil (Cremophor RH 40® by BASF-HLB-13), lauryl macrogol-32 glyceride (Gelucire 44/14® by Gattefosse-HLB-14) stearoyl macrogol glyceride (Gelucire 50/13® by Gattefosse-HLB-13), PEG-20 sorbitan monolaurate (Tween 20® by ICI-HLB-17), PEG-4 lauryl ether (Brij 30® by ICI-HLB-9.7), polyoxyethylene-polyoxypropylene block copolymer (Lutrol® series BASF having different HLB ranging from 15-30), Sodium lauryl sulphate (HLB-40), polyethylene glycol (Carbowax® by DOW), d-α-tocopheryl polyethylene glycol 1000 succinate (Vitamin E TPGS® by Eastman-HLB-15), or mixtures thereof.

The solubilizers can also include pH modifiers such as buffers, amino acids, and amino acid sugars.

In any of the above Valsartan compositions, Valsartan can be present in the form of physical blend, solid dispersion, solid solution, or complex with the solubility enhancing agent. Different processes can be employed to prepare the composition of Valsartan with the solubility enhancing agents. It is contemplated within the scope of the invention that the processes can include, but is not limited to, solubilization using melt granulation, solvent treatment, physical mixing, or spray drying of the dissolved in a solvent with a solubility enhancing agent.

In another illustrative embodiment, the solubilized Valsartan can be incorporated in liquid form into a capsule. In this embodiment. Valsartan mixed with a molten solubility enhancing agent is filled into capsules with or without other excipients. The content of the capsule can remain in liquid or semisolid state during shelf life or the liquid filled into the capsule can set to form a solid mass inside capsule. Optionally excipients such as disintegrants, lubricants, or diluents, can be included in the formulation.

In another illustrative embodiment, the solubilized Valsartan can be dispersed in an excipient, such as microcrystalline cellulose, lactose, mannitol, calcium silicate, magnesium aluminometasilicate (Neusillin) or any other excipient that is generally employed in oral dosage forms. The dispersed mixture can be filled into a capsule or compressed into a tablet.

In another illustrative embodiment, the solubilized Valsartan can be incorporated into a sustained release formulation or a gastroretentive pulsatile pharmaceutical delivery system for sustained release. The solubility enhancing agent ensures better control over the release profile and also complete release of the drug in the desired time interval.

In a further illustrative embodiment, the solubilized Valsartan can be incorporated into a sustained release formulation comprising one or more polymeric or non-polymeric is release retardants. Examples of polymers that can be used include, but are not limited to, polyalkylene oxides, cellulosic polymers, acrylic acid, methacrylic acid polymers, and esters thereof, maleic anhydride polymers, polymaleic acid, poly(acrylamides), poly(olefinic alcohol)s, poly(N-vinyl lactams), polyols, polyoxyethylated saccharides, polyoxazolines, polyvinylamines, polyvinylacetates, polyimines, starch and starch-based polymers, polyurethane hydrogels, chitosan, polysaccharide gums, zein, shellac-based polymers, polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch and polyvinyl alcohol, copolymers, and mixtures thereof.

Solubility enhancing agents used in the composition will increase solubility and dissolution of Valsartan, particularly Valsartan that is in an acidic or weakly acidic environment. In one embodiment, the immediate release component comprising the composition provides at least 40% dissolution in an acidic and weakly acidic environment. The solubility enhancement and a higher dissolution in acidic and weakly acidic environment results in more drug permeating through the GI membrane which leads to increased bioavailability. This increase in solubility also results in a pH independent drug release profile for a drug that is having pH dependent solubility. This invention also reduces inter- and intra-patient variability in drug absorption.

The present invention provides oral solid dosage forms of Valsartan that are about 1.1 to 6 times more bioavailable than the conventional immediate release dosage forms. The increase in bioavailability is evident from the decrease in Tₘₐₓ (time to reach maximum blood concentration), and the increase in Cₘₐₓ (the maximum blood concentration), AUC₀₋ₜ, and AUC_{0-∞} (the extent of absorption, or area under the blood concentration v. time curve). Due to the increase in relative bioavailability, the novel composition will also be able to reduce the variability typically associated with Valsartan. This composition can also achieve peak plasma concentration in less than 4 hours, preferably in less than 3 hours, and more preferably in less than 2 hours. The achieved Tₘₐₓ value is also faster than the conventional immediate release formulation.

Surprisingly, it has been found that a combination of a Valsartan or its salt with certain solubility enhancing agents results in increased solubility and improved dissolution rates over a wide pH range leading to an improved bioavailability compared to the marketed presentation.

### Methods of Treatment

The present invention also relates to a method for the treatment or prevention of a condition or disease using the gastroretentive pulsatile pharmaceutical delivery systems or the pulsatile pharmaceutical delivery systems of the invention. In certain preferred embodiments, the gastroretentive pulsatile pharmaceutical delivery system is orally administered. Upon oral administration an immediate release pulse of Valsartan is released from the delivery system followed by at least a second pulse of Valsartan. In certain further embodiments, the delivery system is concomitantly or sequentially administered with a second agent capable of delaying gastric emptying.

In certain embodiments, the diseases or disorders in a subject to be treated include hypertension, (acute and chronic) congestive heart failure, left ventricular dysfunction and hypertrophic cardiomyopathy, myocardial infarction and its sequelae, supraventricular and ventricular arrhythmias, atrial fibrillation or atrial flutter, atherosclerosis, angina (whether stable or unstable), renal insufficiency (diabetic and non-diabetic), heart failure, angina pectoris, diabetes, hypertension in patients with NIDDM, secondary aldosteronism, primary and secondary pulmonary hyperaldosteronism, primary and pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine. Raynaud's disease, luminal hyperplasia, cognitive dysfunction (such as Alzheimer's), and stroke, comprising administering a pulsatile pharmaceutical delivery system or a gastroretentive pulsatile pharmaceutical delivery system, as described above, to a mammal in need of such treatment.

The gastroretentive delivery systems of the invention are also suitable for veterinary use, for the treatment of mammals, particularly domesticated animals and pets.

The gastroretentive pulsatile pharmaceutical delivery systems and the pulsatile pharmaceutical pulsatile pharmaceutical delivery systems of the present invention can be used to treat diseases and disorders disclosed in WO 04083192, WO04087681, WO03066606, U.S. Pat. No. 6,485,745, US Patent Application Publication No. 2002/0132839 and US Patent Application Publication No. 2003/0152620, all of which are incorporated by herein in their entirety.

### Method of Synthesis

In any of the above Valsartan compositions, Valsartan can be present in the form of a physical blend, solid dispersion, solid solution, or complex with the solubility enhancing agent. Different processes can be employed to prepare the composition of Valsartan with the solubility enhancing agents. It is contemplated within the scope of the invention that the processes can include, but are not limited to, solubilization using melt granulation, solvent treatment, physical mixing, or spray drying of the dissolved in a solvent with a solubility enhancing agent.

In the case of melt granulation, the solubility enhancing agent is melted. The ARB (e.g., Valsartan) is then added and mixed with the molten mass, and allowed to solidify to form granules that are then separated from each other. In another illustrative embodiment of this system, Valsartan is granulated using a molten solubility enhancing agent. In some cases. Valsartan and the solubility enhancing agent both can be melted together and congealed to room temperature.

In using a solvent treatment method, either the solubility enhancing agents or Valsartan, or both, are dissolved in a solvent which is then evaporated or spray dried. The resultant mass is a blend of Valsartan and solubility enhancing agent, such that the solubility of Valsartan is increased. The solvent employed in this system can be aqueous or non-aqueous.

In the case of physical mixing, Valsartan and the solubility enhancing agent are preferably intimately dry-mixed using a Hobart mixer, a V-blender, or a high shear granulator.

The IR component, from which the IR pulse is released, is obtained using methods of formulation such as roller compaction, wet granulation and melt granulation amongst others. Other formulations can include solid dispersion and micro-emulsions.

The secondary pulse can be pellets or tablets using polymers to retard the release. The secondary pulse can be formulated by treating Valsartan with solubility enhancers, permeation enhancers, or both, such that the absorption of Valsartan is enhanced. In some embodiments dosage forms that exhibit an enhanced absorption of Valsartan or Valsartan salt will show higher release and/or more absorption of Valsartan as compared to other dosage forms with the same or higher amount of drug substance that are not treated with solubility enhancers, permeation enhancers, or both. In some embodiments the same therapeutic effect can be achieved with less Valsartan where Valsartan is enhanced as compared to when it is not. Alternatively, the pulse system can be a combination of the IR pulse and enhanced absorption systems. Such a system can enhance solubility and promote absorption of the un-ionized species.

Controlled release of Valsartan from the formulation may be achieved by designing a gastroretentive system (GR). Typically, a drug absorbed mainly from the stomach and proximal part of the small intestine can benefit from such a system. However, in order to accommodate multiple pulses in a reasonable amount of time, a GR system is conceived. Gastroretentive characteristics can be incorporated into the drug delivery system by techniques such as treating active agent with polymers having specific affinity to bind with gastric mucosa, reducing specific gravity of the dosage form leading to floatation, increasing size of the dosage form such that it is greater than the pyloric diameter, and/or using chemicals that delay gastric emptying, and the like, or a combination of more than one such techniques. In an embodiment, the gastroretentivity of the dosage form composition might also be achieved by delaying the gastric emptying time such as by administration of food.

In certain embodiments, the IR component is formed by blending Valsartan o salt with colloidal anhydrous silica (e.g., AEROSIL), microcrystalline cellulose (e.g., AVICEL), and polyvinylpyrrolidone (e.g., CROSPOVIDONE) to form a mixture. This mixture is then passed through a sieve to form a first blend. In some embodiments the seive can be of mesh size 30. The first blend is then granulated using a suitable roller compactor and mill with a suitable screen to form a second blend. Magnesium stearate is then passed through a seive (e.g., of mesh size 100) and mixed with the second blend to form the IR component.

In certain embodiments, the MR component, which can include one or more DR regions, is formulated from materials that can consist of a hydrophilic matrix, swellable gelled-matrix and solubility enhanced or permeability enhanced Valsartan. Additionally, the swellable gelled-matrix can be incorporated such that the MR component or DR regions can be gastroretentive.

In certain embodiments, the MR component of the can be formulated in form of a tablet or tablet in a capsule, which is formulated as a gastroretentive system. The release retarding material used in forming the MR component or DR regions of the present invention can be a swellable polymer. The active ingredient can be 5% to 95% w/w of the composition, one or more release controlling materials from 2% to 95% w/w of the composition and one or more pharmaceutical excipients from 3% to 80% w/w of the composition. These materials are preferably hydrophilic in nature. These may be natural, semi-synthetic, synthetic or modified. Suitable materials include cellulose and cellulose derivatives such as methylcellulose, ethyl cellulose, hydroxypropyl methylcellulose, hydroxypropyl cellulose, cellulose acetate phthalate, cellulose acetate, cellulose acetate butyrate, cellulose acetate propionate, cellulose acetate trimellitate, cellulose carboxymethyl ethers and their salts, hydroxypropyl methylcellulose phthalate, hydroxypropyl methylcellulose acetate succinate, polyethylene; polyquaternium-1; polyvinyl acetate (homopolymer); polyvinyl acetate phthalate; propylene glycol alginate; polyvinyl methacrylate(PVM)/methacrylic acid(MA) copolymer; polyvinyl pyrrolidone (PVP); PVP/dimethiconylacrylate/polycarbamyl/polyglycol ester; PVP/dimethylaminoethyl methacrylate copolymer; PVP/dimethylaminoethylmethacrylate/polycarbamyl/polyglycol ester; PVP/polycarbamyl polyglycol ester; PVP/vinyl acetate (VA) copolymer; lanolin and lanolin derivatives; glyceryl monostearate; stearic acid; paraffins; beeswax; carnauba wax; tribehenin; polyalkylene polyols like polyethylene glycols; gelatin and gelatin derivatives; alginates; carbomers; polycarbophils; methacrylic acid polymers and copolymers; carrageenans; pectins; chitosans; cyclodextrins; lecithins; natural and synthetic gums containing galactomannans like xanthan gum, tragacanth, acacia, agar, guar gum, karaya gum, locust bean gum, gum arabic, and the like, used either alone or in combination.

In certain embodiments the composition can also comprise other polymers selected to retard or delay the release of the agent (i.e., Valsartan) from the system. The MR component may also comprise a permeation enhancers or solubility enhancers as discussed above.

In certain embodiments the IR component and the MR component may consist of the same or different materials. In formulating the MR component with enhanced Valsartan, a first blend of the MR component is formed by blending Valsartan or its salt, microcrystalline cellulose (e.g., AVICEL), and polyvinylpyrrolidone (e.g., CROSPOVIDONE). The first blend of the MR component is passed through a seive to form a second blend of the MR component. A third blend of the MR component is formed by melting Gellucire or a Vitamin (e.g., E TPGS) and adding microcrystalline cellulose to the melt. The second blend of the MR component is then blended with the third blend to form a fourth blend. The fourth blend is then granulated and passed through a mesh and then blended with magnesium stearate to form a fifth blend.

In some embodiments, the secondary pulse from the MR component can be achieved either by the geometric methods introduced in this invention of by a ternary layer in a tablet or by an additional layer in a tablet or a capsule. The arrangement can also allow third, fourth, or fifth pulses to be released in sequence after the IR component has released the first pulse. In certain embodiments the composition of his system is that of the example given in IA.

In some embodiments the MR component, which includes one or more DR regions, may be formulated with enteric polymers such that, for example, the second, third, fourth, fifth, etc. pulses can be enteric in nature. Suitable polymers for enteric delivery are as discussed above.

In the complexation method, complex of Valsartan can be prepared using different techniques such as ball milling, solvent evaporation method which includes spray drying and lyophilization process, slurry method, paste method, etc.

It is contemplated within the scope of the invention that a combination of the aforementioned processes can be employed. For example, a combination of hot melt process, physical mixing, and solvent treatment methods can be employed. In this case, Valsartan can be initially granulated with one or more molten solubility enhancing agents, which can be further treated with the same or different solubility enhancing agents in a solvent or with simple physical mixing or vice versa. It is also contemplated within the scope of the invention that any process known in the art suitable for making pharmaceutical compositions in general can be employed for the purpose of this invention.

Melt granulation and intimate physical mixture are the most preferred methods for preparing pulsatile delivery forms according to the present invention. The increase in solubility can be determined by studying the actual solubility studies of Valsartan in the presence of the solubility enhancing agent, or by carrying out dissolution studies in an appropriate dissolution medium. The dissolution method is preferred, as it allows the comparison of the rate of dissolution of different formulations by determining the amount of Valsartan dissolved at different time intervals.

In certain embodiments, the IR component is distributed about the MR component, which can include one or more DR regions, such that the IR component encompasses the MR component in layered or axial communication. As used herein the term “distributed about” is meant to represent embodiments wherein the IR component is affixed to the MR component by suitable means for forming a layered tablet, capsule, etc.

In certain embodiments, Valsartan in its enhanced form, unenhanced form or both is distributed throughout the system such that Valsartan is in at least one of the IR component, DR regions or any combination thereof. As used herein, the term “distributed throughout,” is meant to embody those instances where Valsartan is incorporated into the system as defined above. A swellable gelled-matrix is incorporated into the MR component such that the system is gastroretentive. The swellable gelled-matrix can comprise a polymeric hydrogel as discussed above.

The composition can be incorporated in various pharmaceutical dosage forms, including, but not limited to, tablets that disintegrate in stomach, tablets that can disintegrate in the mouth, tablets that can disintegrate by effervescence in a liquid (water), tablets that can be dispersed in a liquid (such as water), coated tablets, powders of given doses packaged in sachets, suspensions, gelatin capsules, soft gelatin capsules, semisolid dosage forms, and other drug delivery systems.

The preferred dosage form of the present invention is a solid dosage form, preferably a tablet that can vary in shape, including, but not limited to, oval, triangle, almond, peanut, parallelogram, pentagonal. It is contemplated within the scope of the invention that the dosage form could be encapsulated.

Tablets in accordance with the invention can be manufactured using conventional techniques of tableting known in the art, such as, but not limited to, direct compression, wet granulation, dry granulation, or extrusion/melt granulation.

The dosage form according to the invention can include excipients conventionally known in the art such as fillers, binders and lubricants. Fillers, such as, but not limited to, lactose monohydrate, microcrystalline cellulose, dicalcium phosphate, calcium silicate, magnesium aluminometasilicate (Neusillin), or the like, can be used. Binders, such as, but not limited to, polyvinyl pyrrolidone (PVP), copovidone, or the like, can be used. Lubricants, such as, but not limited to, Aerosil-200, magnesium stearate, hydrogenated vegetable oils, triglycerides of stearic acid, palmitic acid, or the like, can be utilized.

Valsartan can be treated with lipid carriers to form a Valsartan composition. The lipid carriers can include fatty alcohols, glycerol fatty acid esters, acetylated glycerol fatty acid esters, lower alcohol fatty acids esters, propylene glycol fatty acid esters, sorbitan fatty acid esters, polyethylene glycol sorbitan fatty acid esters, sterols and sterol derivatives, polyoxyethylated sterols or sterol derivatives, polyethylene glycol alkyl ethers, sugar esters, sugar ethers, lactic acid derivatives of mono- or di-glycerides, hydrophobic transesterification products of a polyol with at least one member of the group consisting of glycerides, vegetable oils, hydrogenated vegetable oils, fatty acids and sterols, oil-soluble vitamins/vitamin derivatives, polyethylene glycol (PEG) sorbitan fatty acid esters, PEG glycerol fatty acid esters, polyglycerized fatty acid, polyoxyethylene-polyoxypropylene block copolymers, sorbitan fatty acid esters, d-α-tocopheryl polyethylene glycol 1000 succinate (Vitamin E TPGS® by Eastman-HLB-15), or mixtures thereof.

In one illustrative embodiment, the dosage form can optionally be coated. Surface coatings can be employed for aesthetic purposes or for dimensionally stabilizing the compressed dosage form. The surface coating can be carried out using any conventional coating agent which is suitable for oral use. The coating can be carried out using any conventional technique employing conventional ingredients. A surface coating can, for example, be obtained using a quick-dissolving film using conventional polymers, such as hydroxypropyl methyl cellulose, hydroxypropyl cellulose, carboxymethyl cellulose, polyvinyl alcohol poly methacrylates, or the like.

In a further illustrative embodiment, a solid pharmaceutical composition in the form of a multilayer or multi-component system for oral administration can be adapted to deliver a first active pharmaceutical agent from a first layer or component immediately upon administration or upon reaching the gastrointestinal tract, and to deliver a second pharmaceutical agent, which can be same or different from the first agent, from a second layer or component, in a controlled manner over a specific time period.

### EXAMPLES

In order to further illustrate the present invention and the advantages thereof, the following specific examples are given, it being understood that same are intended only as illustrative and in nowise limitative.

### Example 1

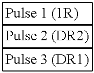

1A) Pulse 1: Immediate Release Component:

| Serial | Ingredients | mg/unit |
|---|---|---|
| 1 | Valsartan or its salt | 80 |
| 2 | colloidal anhydrous silica AEROSIL | 1.5 |
| 3 | microcrystalline cellulose AVICEL | 31.5-34.0 |
| 4 | polyvinylpyrrolidone CROSPOVIDONE | 20 |
| 5 | magnesium stearate | 4.5 |
| | Total | 150 |

AEROSIL is available from Evonik Industries. AVICEL is available from FMC Inc and CROSPOVIDONE is available from ISP Inc.

Preparation of the immediate release region comprises the steps of (i) blending 1, 2, 3 and 4 and passing them through a sieve of mesh size 30 (ii) granulating the blend using a suitable roller compactor and milling using a suitable screen and (iii) passing the resulting material through a sieve of mesh size 100 and blending with the products of step (ii).

1B) Pulse 2: Delayed Release Region: This layer can consist of a hydrophilic matrix, a solubility enhanced or permeability enhanced Valsartan composition, or a combination thereof. Additionally, this layer can be capable of gastroretention using polymers or other swellable materials.

The modified release part of the pharmaceutical delivery system of the Valsartan composition is preferably formulated in the form of a tablet or tablet in a capsule, which is formulated as a gastroretentive system. The release modifying material used in the composition of the present invention is a swellable polymer, described herein. The active ingredient can be 5% to 95% w/w of the composition, one or more release modifying ingredients from 2% to 95% w/w of the composition and one or more pharmaceutical excipients from 3% to 80% w/w of the composition.

The release modifying ingredients of the present invention are preferably hydrophilic in nature. These can be natural, semi-synthetic, synthetic or modified and can be selected from the list of modifying materials disclosed above.

The release modifying ingredient can also be a permeation enhancer, selected from, but not limited to, a group comprising Vitamin E tocopheryl propylene glycol succinate (Vitamin E TPGS), piperine, a lipid, or a surfactant, or their mixtures.

### Example 2

1C) Extended/Delayed Release Layer:

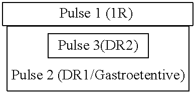

The secondary pulse can be achieved either by the methods introduced in this invention or by a ternary layer in a tablet or by an additional layer in a tablet or a capsule. The arrangement allows an immediate release component to be introduced at the end of the GR phase. The composition of this system is that of the example given in 1A.

The secondary pulse can optionally be enteric in nature. Representative examples of enteric polymers useful in the invention, as described above, include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methamethacrylate copolymers and shellac. These polymers can be used as a dry powder or an aqueous dispersion. Some commercially available materials that can be used are methacrylic acid copolymers sold under the trademark Eudragit (L100, S100, L30D) manufactured by Rhom Pharma, Cellacefate (cellulose acetate phthalate) from Eastman Chemical Co., Aquateric (cellulose acetate phthalate aqueous dispersion) from FMC Corp., and Aqoat (hydroxypropyl methylcellulose acetate succinate aqueous dispersion) from Shin Etsu K.K.

### Example 3

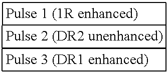

### 2A) Pulse 1: Enhanced Immediate Release Layer:

| Serial | Ingredient | mg/unit |
|---|---|---|
| 1 | Valsartan or its salt | 80 |
| 2 | Gellucire or Vitamin E TPGS | 50-100 |
| 3 | microcrystalline cellulose AVICEL | 20-40 |
| 4 | Surfactant (Labrasol) | 15-25 |
| 5 | polyvinylpyrrolidone CROSPOVIDONE | 15-25 |
| 6 | magnesium stearate | 5 |
| | Total | 185-275 |

E TPGS is available from Eastman Chemical Company.

Preparation of this embodiment comprises the steps of (i) blending 1, 3 and 5 and passing them through a sieve of mesh size 30 (ii) melting 2 and adding 3 (iii) adding the blend obtained from step (i) and mixing to obtain a slurry (iv) cooling down the slurry, (v) granulating the blend from step (ii) by passing through a mesh and (vi) blending the above granules with 6.

### 2B) Gastroretentive Component:

As described herein, this layer can consist of a hydrophilic swellable gelled matrix incorporated with a solubility enhanced or permeability enhanced Valsartan composition or a combination thereof. Additionally, this layer can be capable of gastroretention using the swellable material.

The modified release component of the pharmaceutical delivery systems described herein are preferably formulated in the form of a tablet or tablet in a capsule, which is formulated as a gastroretentive system. The release modifying material used in the composition of the present invention is a swellable polymer. The active ingredient can be 5% to 95% w/w of the composition, one or more release controlling materials from 2% to 95% w/w of the composition and one or more pharmaceutical excipients from 3% to 80% w/w of the composition.

### 2C) Extended/Delayed Release Layer:

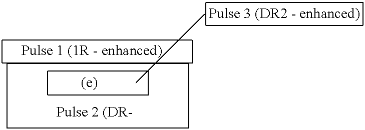

The secondary pulse can be achieved either by the geometric methods introduced in this invention or by a ternary layer in a tablet or by an additional layer in a tablet or a capsule. The arrangement allows the IR pulse to be introduced at the end of the GR phase. The composition of this system is that of the example given in IA.

Other possible examples include the following:

1. IR-DR2-DR1 (Enhanced)

2. IR (Enhanced)-DR2-DR1 (Enhanced)

3. IR (Enhanced)-DR2 (Enhanced)-DR1 (Enhanced)

### Example 4

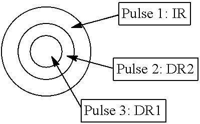

Example 4 displays a schematic of the pulsatile pharmaceutical delivery system of the invention wherein the components are in axial communication. In certain other embodiments the system includes additional delayed release regions and/or additional immediate release components. In certain other embodiments a swellable gelled-matrix is incorporated, as described above, such that the system is retained in the gastrointestinal tract beyond at least one period of gastric emptying. In certain embodiments, Valsartan is incorporated in its enhanced or unenchanced form in any one or any combination of the components.

### Example 5

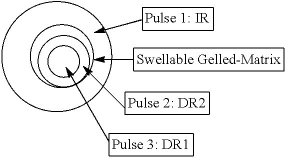

Example 5 displays a schematic of the pulsatile pharmaceutical delivery system of the invention wherein the components are in axial communication with each other and wherein a swellable gelled matrix is disposed between the IR component and DR2 such that the system is gastroretentive. In some other embodiments the swellable gelled matrix is disposed between DR1 and DR2.

1-34. (canceled)

35. A gastroretentive pulsatile pharmaceutical delivery system for the sustained delivery of Valsartan to a subject, comprising:
an immediate release (IR) component that provides a first pulse of Valsartan; and
a modified release (MR) component that provides at least one additional pulse of Valsartan;
wherein Valsartan is in a solubility enhanced form such that the pulsatile delivery of Valsartan occurs in a therapeutically effective and gastroretentive manner.

36. The gastroretentive pulsatile pharmaceutical delivery system of claim 35, wherein Valsartan is incorporated into at least one of the IR or MR components, and wherein the IR and MR components are in axial or layered communication with each other.

37. The gastroretentive pulsatile pharmaceutical delivery system of claim 36, wherein the system is gastroretentive such that it is sustained in the gastrointestinal tract beyond at least one period of gastric emptying.

38. The gastroretentive pulsatile pharmaceutical delivery system of claim 36, wherein the MR component is multi-regioned and comprises a first delayed release region and a second delayed release region, wherein the second delayed release region is disposed between the first delayed release region and the IR component, such that Valsartan is delivered from the MR component in at least two pulses, and
wherein the IR component, the first delayed release region and the second delayed release region are in axial or layered communication with each other.

39. The gastroretentive pulsatile pharmaceutical delivery system of claim 38, wherein the first delayed release region and the second delayed release region respectively comprise a first swellable gelled matrix and a second swellable gelled matrix such that upon contact with the gastric milieu the first swellable gelled matrix or the second swellable gelled matrix or both, expands beyond the size of the diameter of the plyorous sphincter such that the device remains in the gastrointestinal tract beyond at least one period of gastric emptying.

40. The gastroretentive pulsatile pharmaceutical delivery system of claim 39, wherein the IR component comprises at least one of a pressed powder infused with Valsartan, a polymer infused with Valsartan, or a polymer that entraps Valsartan between the polymer and the second gelled matrix.

41. The gastroretentive pulsatile pharmaceutical delivery system of claim 39, wherein Valsartan is infused into the first swellable gelled matrix or is encircled by the first swellable gelled matrix.

42. The gastroretentive pulsatile pharmaceutical delivery system of claim 39, wherein Valsartan is infused into the second swellable gelled matrix or is entrapped between the second swellable gelled matrix and the first swellable gelled matrix.

43. The gastroretentive pulsatile pharmaceutical delivery system of claim 41, wherein Valsartan is encircled by the first gelled matrix and wherein Valsartan is in a form that includes a pressed powder, granules, beads, a gel, a liquid or any combination thereof.

44. The gastroretentive pulsatile pharmaceutical delivery system of claim 42, wherein Valsartan is in a form that includes pressed powder, granules, beads, a gel, a liquid or any combination thereof.

45. The gastroretentive pulsatile pharmaceutical delivery system of claim 35, wherein Valsartan is released from the IR component substantially immediately upon oral administration and wherein Valsartan is released from the modified release component at some time subsequent to the release from the IR component.

46. The gastroretentive pulsatile pharmaceutical delivery system of claim 45, wherein the second delayed release component is situated between the first delayed release component and the IR component in axial or layered communication,
wherein a first pulse of Valsartan is released from the IR component substantially immediately upon oral administration;
a second pulse of Valsartan is released from the second delayed release region at some intermediate time;
a third pulse of Valsartan is released from the first delayed release region at some time subsequent to the second pulse.

47. The gastroretentive pulsatile pharmaceutical delivery system of claim 35, wherein Valsartan is treated with a solubility enhancer.

48. A method of treating high blood pressure, congestive heart failure or postmyocardial infarction in a subject in need thereof, comprising orally administering the pharmaceutical delivery system of claim 35 to the subject, such that high blood pressure, congestive heart failure or post-myocardial infarction is treated.

49. A method of preparing a gastroretentive pulsatile pharmaceutical delivery system, comprising;
forming an IR component by blending Valsartan or its salt with colloidal anhydrous silica, microcrystalline cellulose, and polyvinylpyrrolidone to form a mixture and passing the mixture through a seive to form a first blend of the IR component,
granulating the first blend using a suitable roller compactor and mill with a suitable screen to form a second blend of the IR component,
passing magnesium stearate through a seive and mixing with the second blend to form the IR component;
forming a MR component by blending Valsartan or its salt, microcrystalline cellulose, and polyvinylpyrrolidone to form a first blend of the MR component,
passing the first blend through a seive to form a second blend of the MR component,
melting Gellucire or a vitamin and adding microcrystalline cellulose to the melt to form a third blend of the MR component,
blending the second blend of the MR component with the third blend of the MR component to form a fourth blend of the MR component,
granulating and passing the fourth blend of the MR component through a mesh and then blending the fourth blend with magnesium stearate to form a fifth blend of the MR component,
incorporating a swellable gelled-matrix into the system such that the system is gastroretentive;
distributing the IR component about the MR component such that the IR component encompasses the MR component and the components are in axial or layered communication with each other; and
incorporating Valsartan into the system such that Valsartan is distributed throughout the system in enhanced form, unenhanced form or both.
